(19) United States
(12) Patent Application Publication
(10) Pub. No.: US 2021/0219542 A1
(43) Pub. Date: Jul. 22, 2021

(54) COMPOSITIONS AND METHODS FOR STORAGE OF RED BLOOD CELLS

(71) Applicants: THE REGENTS OF THE UNIVERSITY OF COLORADO, A BODY CORPORATE, DENVER, CO (US); THE BOARD OF REGENTS OF THE UNIVERSITY OF TEXAS SYSTEM, AUSTIN, TX (US)

(72) Inventors: ANGELO D'ALESSANDRO, DENVER, CO (US); TRAVIS NEMKOV, DENVER, CO (US); KIRK HANSEN, DENVER, CO (US); YANG XIA, HOUSTON, TX (US)

(21) Appl. No.: 17/055,479

(22) PCT Filed: May 15, 2019
(86) PCT No.: PCT/US2019/032472

§ 371 Date: Nov. 13, 2020

Related U.S. Application Data
(60) Provisional application No. 62/671,869, filed on May 15, 2018.

(51) Int. Cl. A01N 1/02; A61K 47/02; A61K 47/18; A61K 47/26; A61K 35/14

(52) U.S. Cl. CPC A01N 1/0226; A61K 47/02; A61K 35/14; A61K 47/26; A61K 47/183

(57) ABSTRACT

The invention includes compositions and methods for storage of at least one blood component of blood in a blood product. In certain embodiments, the blood component is contacted with a composition comprising adenosine and sphingosine 1-phosphate (S1P) prior to storage.

### CROSS-REFERENCE TO RELATED APPLICATIONS

The present application claims priority under 35 U.S.C. § 119(e) to U.S. Provisional Application No. 62/671,869, filed May 15, 2018, which is hereby incorporated by reference herein in its entirety.

### BACKGROUND OF THE INVENTION

Transfusion of blood products, such as whole blood, blood components, and plasma derivatives, represents a key lifesaving therapy for millions of people worldwide every year. Unfortunately, blood products generally have a very short shelf-life. For example, red blood cells (RBCs), one of the components of blood, have a limited shelf-life of up to 42 days.

Further complicating the efforts of storing blood products, results from retrospective clinical trials have identified a correlation between untoward consequences in certain categories of recipients (e.g., traumatized, critically ill, or perioperative patients) and transfusion of RBCs stored longer than 11-14 days. This correlation can be due to a physiological decline in RBCs during storage, including the impairment of energy and oxidative stress defense metabolism, as well as depletion of metabolites critical to RBC function such as ATP and 2,3-diphosphoglycerate (DPG).

There is thus a need in the art for compositions and methods that improve storage of blood products. The present invention addresses and satisfies this need.

### BRIEF SUMMARY OF THE INVENTION

In one aspect, the invention provides a method of storing at least one blood component. In another aspect, the invention provides a stock composition for storing the at least one blood component. In yet another aspect, the invention provides a method of treating a subject suffering a shock due to trauma and/or hemorrhage. In yet another aspect, the invention comprises a kit comprising any stock composition of the invention and an instructional material for use thereof.

In certain embodiments, the method comprises contacting the at least one blood component, adenosine, and sphingosine 1-phosphate (S1P), so as to form a storage composition.

In certain embodiments, the at least one blood component comprises at least one of a red blood cell (RBC), s white blood cell (WBC), a platelet, a plasma derivative, and plasma.

In certain embodiments, the final concentration of adenosine in the storage composition is about 0.01 μM to about 100 μM.

In certain embodiments, the final concentration of S1P in the storage composition is about 0.01 μM to about 12 μM.

In certain embodiments, at least one of the adenosine and S1P is added to the at least one blood component as a stock composition.

In certain embodiments, the volume of the stock composition used is about 1% to about 10% of volume of the storage composition.

In certain embodiments, the concentration of adenosine in the stock composition is about 0.1 μM to about 10 mM.

In certain embodiments, the concentration of S1P in the stock composition is about 0.1 μM to about 1.2 mM.

In certain embodiments, the contacting extends the initial metabolic phase of the at least one blood component under storage.

In certain embodiments, the contacting elicits a hypoxic-like cellular response from the at least one blood component.

In certain embodiments, the at least one blood component, adenosine, and S1P are contacted at any point during a storage period of about 5 weeks to about 7 weeks.

In certain embodiments, the at least one blood component, adenosine, and S1P are contacted prior to storing the at least one blood component by cryopreservation.

In certain embodiments, the method of the invention further comprises contacting the at least one blood component with an additive solution.

In certain embodiments, the additive solution comprises at least one of saline-adenine-glucose (SAG), saline-adenine-glucose, plus mannitol (SAGM), variations of SAG/SAGM, AS-1, AS-3, AS-5, AS-7, MAP, PAGGSM, E-SOL 5, and PAG3M.

In certain embodiments, the additive solution comprises an anti-coagulant agent. In certain embodiments, the anti-coagulant agent comprises at least one of citrate-phosphate-dextrose (CPD), citrate-phosphate-double dextrose (CP2D), and acid citrate or dextrose (ACD), and citrate-phosphate-dextrose-adenine (CPDA).

In certain embodiments, the storage composition further comprises at least one of NaCl, NaHCO₃, Na₂HPO₄, NaH₂PO₄, citric acid, Na-citrate, adenine, guanosine, dextrose, glucose, mannitol, gluconate, and an anti-coagulant.

In certain embodiment, the method comprises administering to the subject at least one blood component and a therapeutically effective amount of the composition of the invention.

In certain embodiments, the pH of the storage composition ranges from about 5.0 to about 7.0. In other embodiments, the pH of the stock composition ranges from about 5.0 to about 7.0.

In certain embodiments, the subject is a mammal. In other embodiments, the subject is a human.

In certain embodiments, the instructional material provides instructions for using the composition of the invention for storing the at least one blood components in a blood product.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 is a plot illustrating the finding that Adenosine (Ado) and Sphingosine 1-phosphate (S1P) improve RBC glycolysis during storage.

FIG. 2 is a plot illustrating the finding that Ado and S1P increase cellular 2,3-DPG during storage.

FIG. 3 is a plot illustrating the finding that Ado and S1P increase cellular ATP during early storage.

FIG. 4 is a set of plots illustrating the finding that Ado and S1P improve cellular oxidative stress resistance.

FIG. 5 is a plot illustrating the finding that Ado and S1P decrease hypoxanthine production.

FIG. 6 is a set of plots illustrating the finding that Ado and S1P decrease hemolysis.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, selected materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “blood product” refers to any therapeutic substance prepared from blood. Blood products includes whole blood, any blood component, combinations of two or more blood components, a plasma derivative, and combinations of two or more plasma derivatives.

As used herein, the term “blood storage composition” or “blood storage solution” refers to a composition or solution that is useful for storing blood, and/or any components thereof, and/or any combinations of any components thereof, such that the blood, and/or any components thereof, and/or any combinations of any components thereof, can be reasonably used for transfusions, ex vivo procedures, in vivo procedures, in vitro procedures, or any other appropriate use, after storage. In certain embodiments, the blood storage composition or blood storage solution can be used for storing any other bodily fluids, such as those that may contain blood, and/or any components thereof, and/or any combinations of any components thereof. In other embodiments, the blood storage composition can be used for any other biological applications as appropriate.

As used herein, “component of blood” or “blood component” refers to any one of plasma, red blood cell, white blood cell, and/or platelet. Types of white blood cells include, in a non-limiting example, lymphocytes, monocytes, eosinophils, basophils, and neutrophils.

As used herein, “cryopreservation” refers to a process that preserves organelles, cells, tissues, or any other biological constructs by cooling the samples to very low temperatures, for example, but not limited to, about −80° C. using solid carbon dioxide or −196° C. using liquid nitrogen). Cryopreservation includes controlled rate, slow freezing, and/or a flash-freezing process known as vitrification.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains a composition of the invention or be shipped together with a container which contains the composition. Alternatively, the instructional material may be shipped separately from the container with the intention that the instructional material and the composition be used cooperatively by the recipient.

The term “limited toxicity” as used herein, refers to the compositions of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, the term “plasma derivative” refers to a blood component that contains at least a fraction of plasma proteins.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. In certain embodiments, the subject is human. In other embodiments, the subject is a non-human mammal including, for example, livestock and pets, such as ovine, bovine, porcine, canine, feline and murine mammals.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro or ex vivo. In other embodiments, the cells are not cultured in vitro or ex vivo.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions and methods for blood component, such as but not limited to red blood cell (RBC), storage. In certain embodiments, the invention provides an additive solution that extends the initial metabolic phase of the RBC cells under storage conditions encountered in blood banks.

For several decades, compositions for storing blood components have been prepared by suspending the blood components in a nutrient additive solution, which preserves and extends their shelf-life. In case of the red blood cells (RBCs), preparing such suspensions allow for up to 5-7 weeks of refrigerated storage of the RBCs. Nevertheless, during storage, RBCs undergo a complex and progressive accumulation of physicochemical changes, collectively referred to as the RBC storage lesion. Recent clinical studies have identified RBC transfusion as an independent risk factor for increased morbidities and mortalities in certain groups of patients, including trauma, cardiac surgery the critically-ill, and the chronically transfused. Additionally, some of these studies have identified that older stored RBCs are more strongly implicated in poorer outcomes compared to fresher RBCs.

### Compositions

In one aspect, the invention provides a composition for storage of blood components. In certain embodiments, the composition comprises adenosine and/or sphingosine 1-phosphate (S1P).

In certain embodiments, a blood component includes at least one of a red blood cell (RBC), a white blood cell (WBC), a platelet, a plasma derivative, and a plasma.

In certain embodiments, the composition further comprises at least one of NaCl, KCl, MgCl₂, KH₂PO₄, NaHCO₃, Na₂HPO₄, NaH₂PO₄, citric acid, Na-citrate, adenine, guanosine, dextrose, glucose, mannose, fructose, sorbitol, mannitol, gluconate, glycerol, dimethyl sulphoxide (DMSO), hydroxyethyl starch (HES), and an anti-coagulant.

In certain embodiments, the pH of the composition ranges from about 5.0 to about 9.0.

In certain embodiments, the pH of the composition is about 5.0. In certain embodiments, the pH of the composition is about 5.1. In certain embodiments, the pH of the composition is about 5.2. In certain embodiments, the pH of the composition is about 5.3. In certain embodiments, the pH of the composition is about 5.4. In certain embodiments, the pH of the composition is about 5.5. In certain embodiments, the pH of the composition is about 5.6. In certain embodiments, the pH of the composition is about 5.7. In certain embodiments, the pH of the composition is about 5.8. In certain embodiments, the pH of the composition is about 5.9. In certain embodiments, the pH of the composition is about 6.0. In certain embodiments, the pH of the composition is about 6.1. In certain embodiments, the pH of the composition is about 6.2. In certain embodiments, the pH of the composition is about 6.3. In certain embodiments, the pH of the composition is about 6.4. In certain embodiments, the pH of the composition is about 6.5. In certain embodiments, the pH of the composition is about 6.6. In certain embodiments, the pH of the composition is about 6.7. In certain embodiments, the pH of the composition is about 6.8. In certain embodiments, the pH of the composition is about 6.9. In certain embodiments, the pH of the composition is about 7.0. In certain embodiments, the pH of the composition is about 7.1. In certain embodiments, the pH of the composition is about 7.2. In certain embodiments, the pH of the composition is about 7.3. In certain embodiments, the pH of the composition is about 7.4. In certain embodiments, the pH of the composition is about 7.5. In certain embodiments, the pH of the composition is about 7.6. In certain embodiments, the pH of the composition is about 7.7. In certain embodiments, the pH of the composition is about 7.8. In certain embodiments, the pH of the composition is about 7.9. In certain embodiments, the pH of the composition is about 8.0. In certain embodiments, the pH of the composition is about 8.1. In certain embodiments, the pH of the composition is about 8.2. In certain embodiments, the pH of the composition is about 8.3. In certain embodiments, the pH of the composition is about 8.4. In certain embodiments, the pH of the composition is about 8.5. In certain embodiments, the pH of the composition is about 8.6. In certain embodiments, the pH of the composition is about 8.7. In certain embodiments, the pH of the composition is about 8.8. In certain embodiments, the pH of the composition is about 8.9. In certain embodiments, the pH of the composition is about 9.0.

In certain embodiments, the final concentration of adenosine for storing the at least one blood component is about 0.01 μM to about 100 μM.

In certain embodiments, the final concentration of S1P for storing the at least one blood component about 0.01 μM to about 12 μM.

In certain embodiments, the composition of the invention is provided as a concentrated stock composition comprising adenosine and/or S1P. In certain embodiments, the composition of the invention is provided as a concentrated stock composition comprising higher concentrations of adenosine and/or S1P than that required for storing the blood component. In certain embodiments, the concentration of adenosine and/or S1P in the concentrated stock composition is about 10 times to about 100 times higher than that required for the storage of the blood component.

In certain embodiments, the stock solution is physiologically compatible. In certain embodiments, the storage solution is physiologically compatible.

In certain embodiments, the stock solution is isotonic with at least bodily fluid, such as but not limited to blood and/or plasma. In certain embodiments, the storage solution is isotonic with at least bodily fluid, such as but not limited to blood and/or plasma.

In certain embodiments, the concentration of adenosine in the concentrated stock composition is about 0.1 μM to about 1 mM. In certain embodiments, the concentration of adenosine in the concentrated stock composition is about 0.2 μM to about 2 mM. In certain embodiments, the concentration of adenosine in the concentrated stock composition is about 0.3 μM to about 3 mM. In certain embodiments, the concentration of adenosine in the concentrated stock composition is about 0.4 μM to about 4 mM. In certain embodiments, the concentration of adenosine in the concentrated stock composition is about 0.5 μM to about 5 mM. In certain embodiments, the concentration of adenosine in the concentrated stock composition is about 0.6 μM to about 6 mM. In certain embodiments, the concentration of adenosine in the concentrated stock composition is about 0.7 μM to about 7 mM. In certain embodiments, the concentration of adenosine in the concentrated stock composition is about 0.8 μM to about 8 mM. In certain embodiments, the concentration of adenosine in the concentrated stock composition is about 0.9 μM to about 9 mM. In certain embodiments, the concentration of adenosine in the concentrated stock composition is about 1 μM to about 10 mM.

In certain embodiments, the concentration of S1P in the concentrated stock composition is about 0.1 μM to about 120 μM. In certain embodiments, the concentration of S1P in the concentrated stock composition is about 0.2 μM to about 240 μM. In certain embodiments, the concentration of S1P in the concentrated stock composition is about 0.3 μM to about 360 μM. In certain embodiments, the concentration of S1P in the concentrated stock composition is about 0.4 μM to about 480 μM. In certain embodiments, the concentration of S1P in the concentrated stock composition is about 0.5 μM to about 600 μM. In certain embodiments, the concentration of S1P in the concentrated stock composition is about 0.6 μM to about 720 μM. In certain embodiments, the concentration of S1P in the concentrated stock composition is about 0.7 μM to about 840 μM. In certain embodiments, the concentration of S1P in the concentrated stock composition is about 0.8 μM to about 960 μM. In certain embodiments, the concentration of S1P in the concentrated stock composition is about 0.9 μM to about 1.08 mM. In certain embodiments, the concentration of S1P in the concentrated stock composition is about 1 mM to about 1.2 mM.

In certain embodiments, the concentration of adenosine in the concentrated stock composition is about 0.1 μM. In certain embodiments, the concentration of adenosine in the concentrated stock composition is about 0.2 μM. In certain embodiments, the concentration of adenosine in the concentrated stock composition is about 0.3 μM. In certain embodiments, the concentration of adenosine in the concentrated stock composition is about 0.4 μM. In certain embodiments, the concentration of adenosine in the concentrated stock composition is about 0.5 μM. In certain embodiments, the concentration of adenosine in the concentrated stock composition is about 0.6 μM. In certain embodiments, the concentration of adenosine in the concentrated stock composition is about 0.7 μM. In certain embodiments, the concentration of adenosine in the concentrated stock composition is about 0.8 μM. In certain embodiments, the concentration of adenosine in the concentrated stock composition is about 0.9 μM. In certain embodiments, the concentration of adenosine in the concentrated stock composition is about 1 μM. In certain embodiments, the concentration of adenosine in the concentrated stock composition is about 2 μM. In certain embodiments, the concentration of adenosine in the concentrated stock composition is about 3 μM. In certain embodiments, the concentration of adenosine in the concentrated stock composition is about 4 μM. In certain embodiments, the concentration of adenosine in the concentrated stock composition is about 5 μM. In certain embodiments, the concentration of adenosine in the concentrated stock composition is about 6 μM. In certain embodiments, the concentration of adenosine in the concentrated stock composition is about 7 μM. In certain embodiments, the concentration of adenosine in the concentrated stock composition is about 8 μM. In certain embodiments, the concentration of adenosine in the concentrated stock composition is about 9 μM. In certain embodiments, the concentration of adenosine in the concentrated stock composition is about 10 μM. In certain embodiments, the concentration of adenosine in the concentrated stock composition is about 20 μM. In certain embodiments, the concentration of adenosine in the concentrated stock composition is about 30 μM. In certain embodiments, the concentration of adenosine in the concentrated stock composition is about 40 μM. In certain embodiments, the concentration of adenosine in the concentrated stock composition is about 50 μM. In certain embodiments, the concentration of adenosine in the concentrated stock composition is about 60 μM. In certain embodiments, the concentration of adenosine in the concentrated stock composition is about 70 μM. In certain embodiments, the concentration of adenosine in the concentrated stock composition is about 80 μM. In certain embodiments, the concentration of adenosine in the concentrated stock composition is about 90 μM. In certain embodiments, the concentration of adenosine in the concentrated stock composition is about 100 μM. In certain embodiments, the concentration of adenosine in the concentrated stock composition is about 200 μM. In certain embodiments, the concentration of adenosine in the concentrated stock composition is about 300 μM. In certain embodiments, the concentration of adenosine in the concentrated stock composition is about 400 μM. In certain embodiments, the concentration of adenosine in the concentrated stock composition is about 500 μM. In certain embodiments, the concentration of adenosine in the concentrated stock composition is about 600 μM. In certain embodiments, the concentration of adenosine in the concentrated stock composition is about 700 μM. In certain embodiments, the concentration of adenosine in the concentrated stock composition is about 800 μM. In certain embodiments, the concentration of adenosine in the concentrated stock composition is about 900 μM. In certain embodiments, the concentration of adenosine in the concentrated stock composition is about 1 mM. In certain embodiments, the concentration of adenosine in the concentrated stock composition is about 1.5 mM. In certain embodiments, the concentration of adenosine in the concentrated stock composition is about 2 mM. In certain embodiments, the concentration of adenosine in the concentrated stock composition is about 2.5 mM. In certain embodiments, the concentration of adenosine in the concentrated stock composition is about 3 mM. In certain embodiments, the concentration of adenosine in the concentrated stock composition is about 3.5 mM. In certain embodiments, the concentration of adenosine in the concentrated stock composition is about 4 mM. In certain embodiments, the concentration of adenosine in the concentrated stock composition is about 4.5 mM. In certain embodiments, the concentration of adenosine in the concentrated stock composition is about 5 mM. In certain embodiments, the concentration of adenosine in the concentrated stock composition is about 5.5 mM. In certain embodiments, the concentration of adenosine in the concentrated stock composition is about 6 mM. In certain embodiments, the concentration of adenosine in the concentrated stock composition is about 6.5 mM. In certain embodiments, the concentration of adenosine in the concentrated stock composition is about 7 mM. In certain embodiments, the concentration of adenosine in the concentrated stock composition is about 7.5 mM. In certain embodiments, the concentration of adenosine in the concentrated stock composition is about 8 mM. In certain embodiments, the concentration of adenosine in the concentrated stock composition is about 8.5 mM. In certain embodiments, the concentration of adenosine in the concentrated stock composition is about 9 mM. In certain embodiments, the concentration of adenosine in the concentrated stock composition is about 9.5 mM. In certain embodiments, the concentration of adenosine in the concentrated stock composition is about 10 mM.

In certain embodiments, the concentration of S1P in the concentrated stock composition is about 0.1 μM. In certain embodiments, the concentration of S1P in the concentrated stock composition is about 0.2 μM. In certain embodiments, the concentration of S1P in the concentrated stock composition is about 0.3 μM. In certain embodiments, the concentration of S1P in the concentrated stock composition is about 0.4 μM. In certain embodiments, the concentration of S1P in the concentrated stock composition is about 0.5 μM. In certain embodiments, the concentration of S1P in the concentrated stock composition is about 0.6 μM. In certain embodiments, the concentration of S1P in the concentrated stock composition is about 0.7 μM. In certain embodiments, the concentration of S1P in the concentrated stock composition is about 0.8 μM. In certain embodiments, the concentration of S1P in the concentrated stock composition is about 0.9 μM. In certain embodiments, the concentration of S1P in the concentrated stock composition is about 1 μM. In certain embodiments, the concentration of S1P in the concentrated stock composition is about 2 μM. In certain embodiments, the concentration of S1P in the concentrated stock composition is about 3 μM. In certain embodiments, the concentration of S1P in the concentrated stock composition is about 4 μM. In certain embodiments, the concentration of S1P in the concentrated stock composition is about 5 μM. In certain embodiments, the concentration of S1P in the concentrated stock composition is about 6 μM. In certain embodiments, the concentration of S1P in the concentrated stock composition is about 7 μM. In certain embodiments, the concentration of S1P in the concentrated stock composition is about 8 μM. In certain embodiments, the concentration of S1P in the concentrated stock composition is about 9 μM. In certain embodiments, the concentration of S1P in the concentrated stock composition is about 10 μM. In certain embodiments, the concentration of S1P in the concentrated stock composition is about 20 μM. In certain embodiments, the concentration of S1P in the concentrated stock composition is about 30 μM. In certain embodiments, the concentration of S1P in the concentrated stock composition is about 40 μM. In certain embodiments, the concentration of S1P in the concentrated stock composition is about 50 μM. In certain embodiments, the concentration of S1P in the concentrated stock composition is about 60 μM. In certain embodiments, the concentration of S1P in the concentrated stock composition is about 70 μM. In certain embodiments, the concentration of S1P in the concentrated stock composition is about 80 μM. In certain embodiments, the concentration of S1P in the concentrated stock composition is about 90 μM. In certain embodiments, the concentration of S1P in the concentrated stock composition is about 100 μM. In certain embodiments, the concentration of S1P in the concentrated stock composition is about 200 μM. In certain embodiments, the concentration of S1P in the concentrated stock composition is about 300 μM. In certain embodiments, the concentration of S1P in the concentrated stock composition is about 400 μM. In certain embodiments, the concentration of S1P in the concentrated stock composition is about 500 μM. In certain embodiments, the concentration of S1P in the concentrated stock composition is about 600 μM. In certain embodiments, the concentration of S1P in the concentrated stock composition is about 700 μM. In certain embodiments, the concentration of S1P in the concentrated stock composition is about 800 μM. In certain embodiments, the concentration of S1P in the concentrated stock composition is about 900 μM. In certain embodiments, the concentration of S1P in the concentrated stock composition is about 1 mM. In certain embodiments, the concentration of S1P in the concentrated stock composition is about 1.5 mM. In certain embodiments, the concentration of S1P in the concentrated stock composition is about 2 mM.

In certain embodiments, the composition of the invention is stored in an additive solution selected from an anti-coagulant solution comprising citrate-phosphate-dextrose (CPD), citrate-phosphate-double dextrose (CP2D), acid citrate dextrose (ACD), citrate-phosphate-dextrose-adenine (CPDA) and any variations thereof.

In certain embodiments, the composition of the invention is stored in an anti-coagulant solution comprising citrate-phosphate-dextrose (CPD), citrate-phosphate-double dextrose (CP2D), or anticoagulant citrate dextrose (ACD), citrate-phosphate-dextrose-adenine (CPDA), or any combination thereof.

In certain embodiments, the at least one blood component is contacted with a composition of the invention prior to a long-term storage of the at least one blood component by cryopreservation.

In certain embodiments, the at least one blood component is contacted with the mixture of a blood storage composition of the invention and an additive solution.

In certain embodiments, an additive solution is contacted with the mixture of the at least one blood component and a blood storage composition of the invention.

In certain embodiments, a blood storage composition of the invention is contacted with the mixture of the at least one blood component and an additive solution.

Methods

In another aspect, the invention provides a method of storing blood, and/or at least one blood component, wherein the method comprises contacting the blood, and/or the at least one blood component, adenosine, and S1P, prior to storage. In yet another aspect, the invention includes a method of treating (such as, for example, resuscitating) a subject suffering from a shock due to trauma and/or hemorrhage, the method comprises co-administering at least one blood component and therapeutically effective amount of a composition of the invention to the subject in need thereof.

In certain embodiments, the method comprises contacting the at least one blood component, adenosine, and S1P at any point during a storage period of about 5 weeks to about 7 weeks.

Signaling molecules were identified that modulate the maintenance of metabolic features in RBCs and can thus improve the quality and functionality of stored RBCs. Unique to this additive solution are the inclusion of adenosine and S1P, either independently or in combination. In one embodiment, about 0.01 μM to about 12 μM S1P and 0.01 μM to about 100 μM adenosine are contacted with the components of blood prior to storage, either through original formulation or through the supplementation of a stock solution containing about 0.1 μM to about 120 μM S1P and about 0.1 μM to about 1000 μM adenosine by volume dilution or satellite bag transfer into any additive solution. Without wishing to be limited by theory, the presence of these molecules in the additive solution elicits a hypoxic-like adaptive cellular response that delays the onset of the storage lesion and potentially improves patient outcomes.

In certain embodiments, the composition of the invention is stored in an anti-coagulant solution comprising citrate-phosphate-dextrose (CPD), citrate-phosphate-double dextrose (CP2D), anticoagulant citrate dextrose (ACD), citrate-phosphate-dextrose-adenine (CPDA), and any variations thereof.

The compositions of the invention modulate the maintenance of metabolic features in the blood components and thus improve the quality and functionality of stored blood-components. Since the oldest blood donations are typically transfused first (they can be as old as 42 days), ensuring quality is unaffected or limiting decline in quality during storage is paramount.

In certain embodiments of the invention, adenosine and S1P can be combined with an additive solution. Examples of additive solutions include but are not limited to saline-adenine-glucose (SAG) (Högman et al., New Engl J Med 1978; 299: 1377-82), saline-adenine-glucose plus mannitol (SAGM), variations of SAG/SAGM, AS-1 (Heaton et al., Br J Haematol 1984; 57: 467-78), AS-3 (Simon et al., Transfusion 1987; 27: 178-82), AS-5, MAP (Cicha et al., Vox Sang 2000; 79:75-82), PAGGSM (Walker et al., Beitr Infusionsther 1990; 26: 55-9), bicarbonate-adenine-glucose-phosphate-mannitol (also known as BAGPM; Chillar et al J Lab Clin Med 1977; 89(3): 504-8), variations of BAGPM, AS-7 (also known as SOL-X; Cancelas et al., Transfusion. 2015 March; 55(3):491-8), PAG3M (de Korte et al., Transfusion. 2008 June; 48(6):1081-9), and E-Sol 5(Högman et al., Transfusion. 2002 July; 42(7):824-9).

Certain embodiments of the invention include the combination of adenosine and sphingosine 1-phosphate (S1P) and various other components useful for RBC storage, including but not limited to NaCl, NaHCO₃, Na₂HPO₄, NaH₂PO₄, citric acid, Na-citrate, adenine, guanosine, dextrose, glucose, mannitol, gluconate, and an anti-coagulant (CPD, CP2D, ACD).

In certain embodiments, the subject is a mammal. In certain embodiments, the subject is a human subject. In certain embodiments, the subject is a non-human mammal.

### Kit

In yet another aspect, the invention provides a kit comprising the composition of the invention and an instructional material for use thereof, wherein the instructional material comprises instructions for storing at least one component of blood in a blood product.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

RBC Storage: To ensure sterility, all storage conditions were set up and subsequently sampled in a biosafety cabinet. The RBC-AS-3 suspension was carefully removed from the bag into 3 ml aliquots inside 15 ml conical tubes. Stock concentrations (formulated in house-made AS-3) of ¹³C₅-Ado or d₇-S1P were diluted to final concentrations of 0.1, 1, 10, and 100 μM for Ado or 1, 5, and 10 μM for S1P. Also included was a storage condition consisting of 10 Ado+5 μM S1P. To mimic plasticizer conditions (D'Alessandro et al. (2016) Blood Transfus. 14, 140-144), concentrated diethylhexyl phthalate (DEHP) was added to each tube to a final concentration of 800 though any comparable plasticizer or piece of a blood bag plastic will suffice. The tubes were gently mixed and placed at 4° C. for storage.

On days 2, 8, 11, 15, 22, 29, and 43, the tubes were placed back in a biosafety cabinet and 300 μl were sampled. The tubes were placed back at 4° C., and the samples were centrifuged for 10 minutes at 1500 g, 4° C. Supernatants were separated from cells and frozen until analysis (1^{st} analysis after day 22 sampling).

LC-MS Metabolomics: Metabolomics analyses were performed as previously described (Nemkov et al. (2015) Amino Acids doi:10.1007/s00726-015-2019-9; Nemkov et al. (2017) Rapid Commun. Mass Spectrom. 31, 663-673). Briefly, samples were placed on ice and diluted with 9 volumes of methanol:acetonitrile:water (5:3:2, v:v) for RBC, or with 24 volumes of methanol:acetonitrile:water (5:3:2, v:v) for supernatants. Suspensions were vortexed continuously for 30 min at 4° C. Insoluble material was removed by centrifugation at 18,000 g for 10 min at 4° C. and supernatants were isolated for metabolomics analysis by UHPLC-MS.

The analytical platform employed a Vanquish UHPLC system (Thermo Fisher Scientific, San Jose, Calif., USA) coupled online to a Q Exactive mass spectrometer (Thermo Fisher Scientific, San Jose, Calif., USA). Samples were resolved over a Kinetex C18 column, 2.1×150 mm, 1.7 μm particle size (Phenomenex, Torrance, Calif., USA) equipped with a guard column (SecurityGuard™ Ultracartridge—UHPLC C18 for 2.1 mm ID Columns—AJO-8782—Phenomenex, Torrance, Calif., USA) using an aqueous phase (A) of water and 0.1% formic acid and a mobile phase (B) of acetonitrile and 0.1% formic acid for positive ion polarity mode, or an aqueous phase (A) of 5% acetonitrile and 1 mM ammonium acetate in water and a mobile phase (B) of 95% acetonitrile and 1 mM ammonium acetate in water for negative ion polarity mode. Samples were eluted from the column using a gradient from 5% to 95% B over 1.6 minutes, followed by an isocratic hold at 95% B for 1.65 minutes, flowed at 450 μl/min and 40° C. The Q Exactive mass spectrometer (Thermo Fisher Scientific, San Jose, Calif., USA) was operated independently in positive or negative ion mode, scanning in Full MS mode (2 μscans) from 60 to 900 m/z at 70,000 resolution, with 4 kV spray voltage, 15 shealth gas, 5 auxiliary gas. Calibration was performed prior to analysis using the Pierce™ Positive and Negative Ion Calibration Solutions (Thermo Fisher Scientific). Acquired data was then converted from .raw to .mzXML file format using Mass Matrix (Cleveland, Ohio, USA). Samples were analyzed in randomized order with a technical mixture injected after every 15 samples to qualify instrument performance. Metabolite assignments, isotopologue distributions, and correction for expected natural abundances of deuterium, ¹³C, and ¹⁵N isotopes were performed using MAVEN (Princeton, N.J., USA). The Q Exactive mass spectrometer (Thermo Fisher Scientific, San Jose, Calif., USA) was operated independently in positive or negative ion mode, scanning in Full MS mode (2 μscans) from 60 to 900 m/z at 70,000 resolution, with 4 kV spray voltage, 15 shealth gas, 5 auxiliary gas. Calibration was performed prior to analysis using the Pierce™ Positive and Negative Ion Calibration Solutions (Thermo Fisher Scientific). Acquired data was then converted from .raw to .mzXML file format using Mass Matrix (Cleveland, Ohio, USA).

The results of the experiments are now described.

### Example 1

Recently discovered was a role for physiological production of adenosine and sphingosine 1-phosphate by human and mouse red blood cells exposed to hypoxia in modulating tissue oxygen off-loading upon exposure to hypoxia in vivo by increasing generation of erythrocyte ATP and 2,3-DPG (D'Alessandro et al., J. Proteome Res, 2016, 15 (10), pp 3883-3895). In the present invention, the implications of these observations were exploited to promote ATP and DPG generation under normoxic conditions by supplementing adenosine and/or sphingosine 1-phosphate to packed red cell storage additives for ex vivo preservation of erythrocyte concentrates in the blood bank.

In order to demonstrate the ability of both adenosine (Ado) and sphingosine 1-phosphate (S1P) to mitigate red blood cell metabolic impairments that contribute to the storage lesion, a freshly drawn, leukoreduced blood unit formulated in AS-3 was acquired. Importantly, the use of a standard processed blood unit is an improvement over previous experiments as this material directly represents a realistic storage strategy.

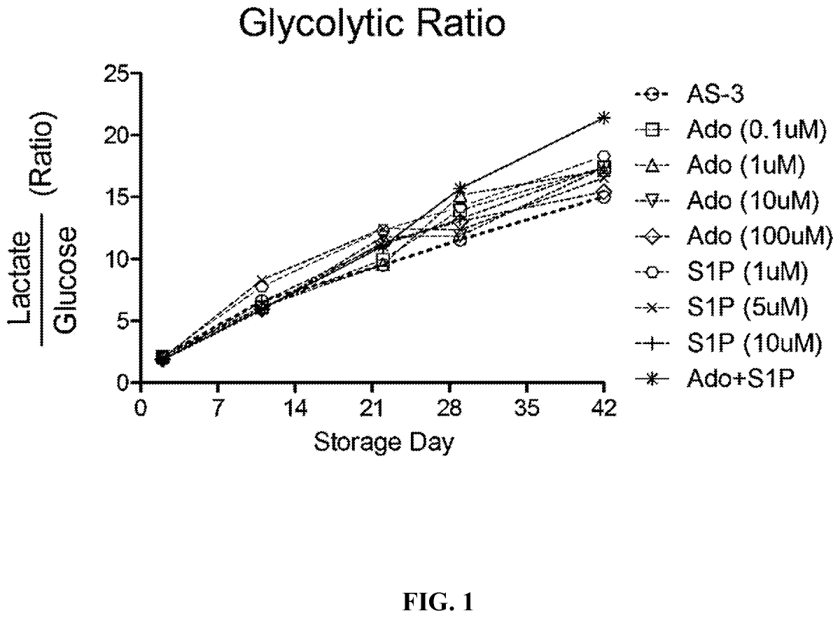

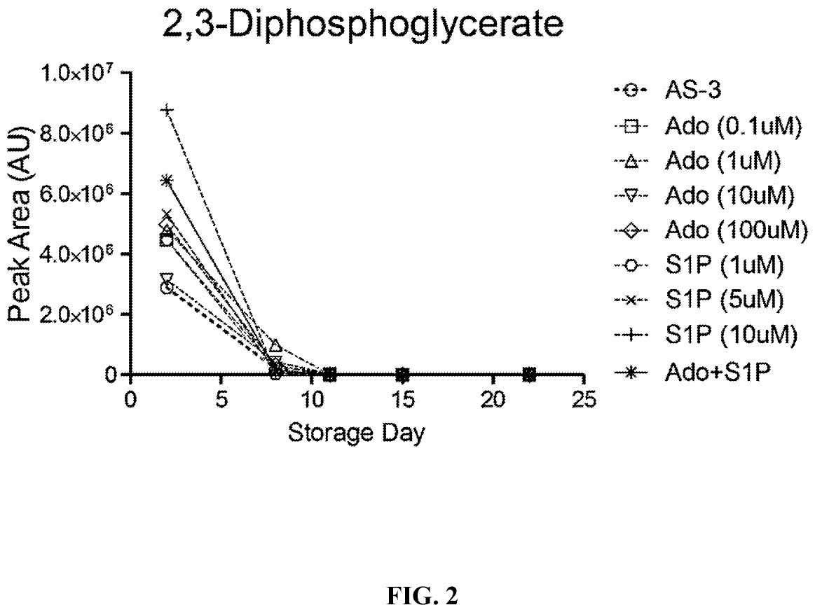

At present, the most widely relied upon metabolites as indicators of RBC metabolic status and functionality are ATP, 2,3-DPG, and lactate. However, storage in the presence of adenosine (Ado) and S1P improved these metabolic parameters, including higher glycolytic activity as measured by the lactate-to-glucose ratio (FIG. 1) and higher 2,3-DPG levels through the first two weeks of storage (FIG. 2). These effects demonstrated the roles of Ado and S1P in metabolic reprogramming of RBCs.

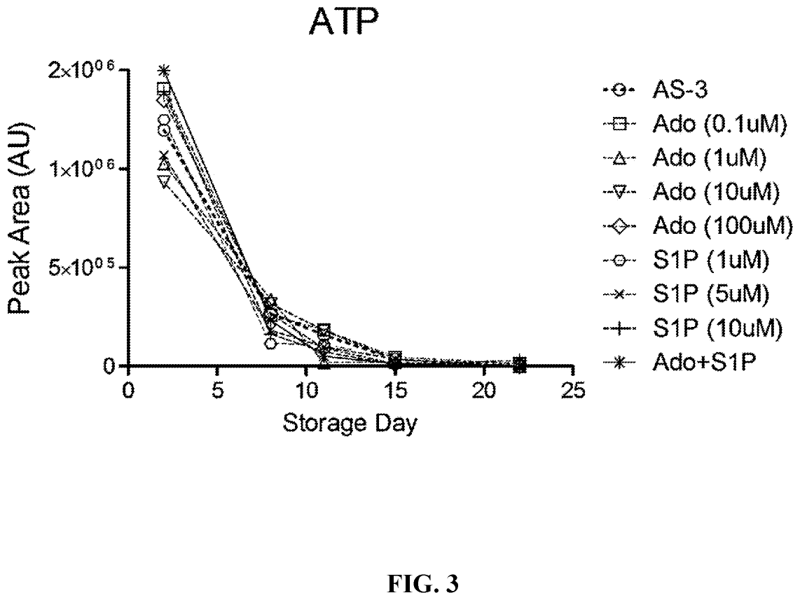

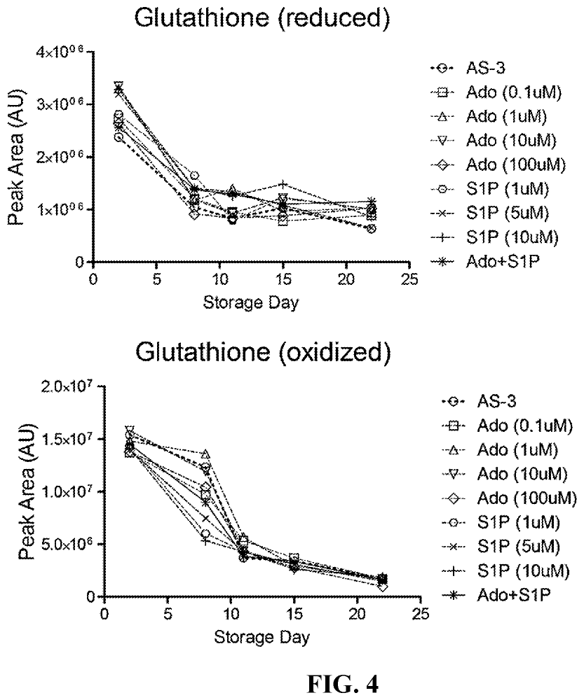

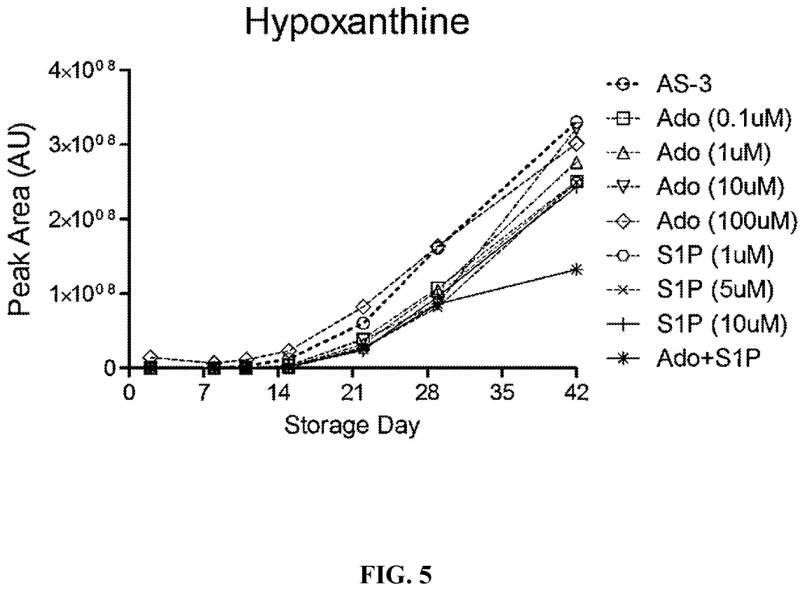

Higher glycolytic activity resulted in improved ATP production through the first week of storage (FIG. 3). Depletion of ATP in the Ado and S1P supplemented storage conditions is thought to be due to increased utilization for glutathione synthesis, and subsequent improvements in capacity to cope with oxidative stress. Indeed, the reduced glutathione levels were increased in the supplemented conditions, and oxidized levels were lower (FIG. 4). Recent work has indicated that the oxidative stress status of RBCs contributes to the production of hypoxanthine through the elevated activity of AMPD and serves as a metabolic marker of the storage lesion. Furthermore, the end-of-storage levels of hypoxanthine appear to inversely correlate with post-transfusion recovery, which is a measure RBC transfusion efficacy due to its correlation with post-transfusion recovery (Nemkov et al., Haematologica, 2018, 103 (2), pp 361-372). Importantly, storage in the presence of Ado and S1P markedly decreased the amount of hypoxanthine both in RBC and supernatants (at least up to a concentration less than 100 μM, at which point Ado becomes a substrate for hypoxanthine production; FIG. 5).

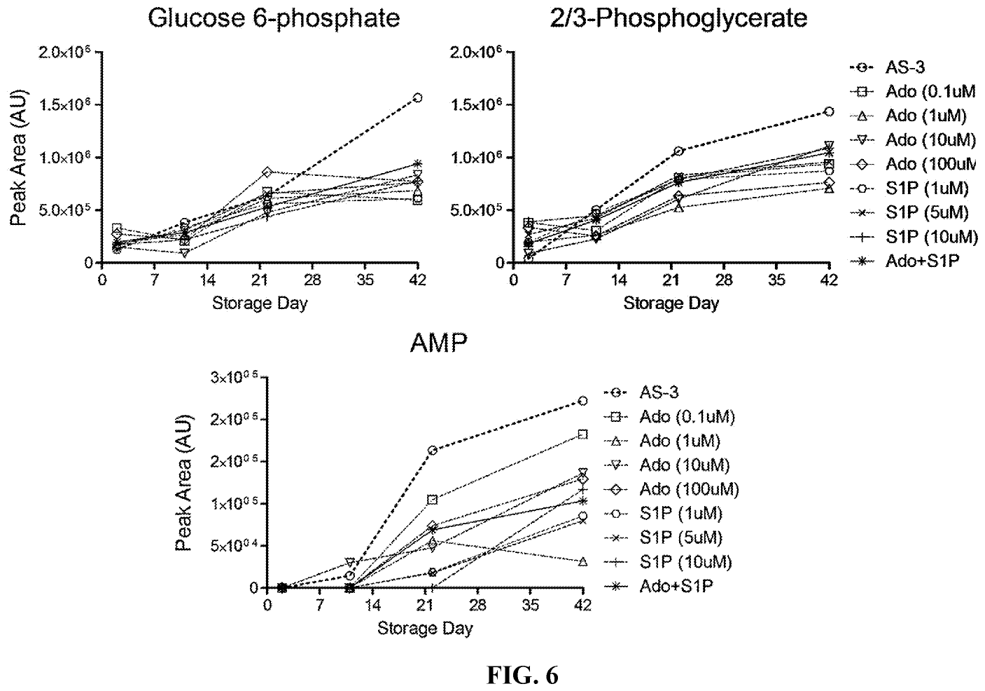

One particular culmination of the storage lesion (i.e. energy depletion and oxidative stress) is hemolysis, or the lysis of RBCs. A standard FDA requirement for RBC storage indicates that fewer than 1% of cells can lyse during storage. The accumulation of phosphate-conjugated carbohydrates and adenylates in the supernatant fraction are indicative of hemolysis as RBCs do not contain transporters for these compounds. While these compounds increase over storage duration in the supernatants of all storage conditions tested here, the levels are significantly lower when RBCs are stored in the presence of Ado and S1P, thus indicating that these compounds mitigate storage hemolysis as well (FIG. 6).

### ENUMERATED EMBODIMENTS

The following exemplary embodiments are provided, the numbering of which is not to be construed as designating levels of importance.

Embodiment 1 provides a method of storing at least one blood component, wherein the method comprises contacting the at least one blood component, adenosine, and sphingosine 1-phosphate (S1P), so as to form a storage composition.

Embodiment 2 provides the method of Embodiment 1, wherein the at least one blood component comprises at least one of a red blood cell (RBC), a white blood cell (WBC), a platelet, a plasma derivative, and plasma.

Embodiment 3 provides the method of any of Embodiments 1-2, wherein the final concentration of adenosine in the storage composition is about 0.01 μM to about 100 μM.

Embodiment 4 provides the method of any of Embodiments 1-3, wherein the final concentration of S1P in the storage composition is about 0.01 μM to about 12 μM.

Embodiment 5 provides the method of any of Embodiments 1-4, wherein at least one of the adenosine and S1P is added to the at least one blood component as a stock composition, wherein the volume of the stock composition used is about 1% to about 10% of volume of the storage composition.

Embodiment 6 provides the method of Embodiment 5, wherein the concentration of adenosine in the stock composition is about 0.1 μM to about 10 mM.

Embodiment 7 provides the method of Embodiment 5, wherein the concentration of S1P in the stock composition is about 0.1 μM to about 1.2 mM.

Embodiment 8 provides the method of any of Embodiments 1-7, wherein the contacting extends the initial metabolic phase of the at least one blood component under storage.

Embodiment 9 provides the method of any of Embodiments 1-8, wherein the contacting elicits a hypoxic-like cellular response from the at least one blood component under storage.

Embodiment 10 provides the method of any of Embodiments 1-9, further comprising contacting the at least one blood component with an additive solution.

Embodiment 11 provides the method of Embodiment 10, wherein the additive solution comprises at least one of saline-adenine-glucose (SAG), saline-adenine-glucose plus mannitol (SAGM), variations of SAG/SAGM, AS-1, AS-3, AS-5, AS-7, MAP, PAGGSM, E-SOL 5, and PAG3M.

Embodiment 12 provides the method of any of Embodiments 10-11, wherein the additive solution comprises an anti-coagulant agent.

Embodiment 13 provides the method of any of Embodiments 10-12, wherein the anti-coagulant agent comprises at least one of citrate-phosphate-dextrose (CPD), citrate-phosphate-double dextrose (CP2D), and acid citrate or dextrose (ACD), and citrate-phosphate-dextrose-adenine (CPDA).

Embodiment 14 provides the method of any of Embodiments 1-13, wherein the storage composition further comprises at least one of NaCl, NaHCO₃, Na₂HPO₄, NaH₂PO₄, citric acid, Na-citrate, adenine, guanosine, dextrose, glucose, mannitol, gluconate, and an anti-coagulant.

Embodiment 15 provides the method of any of Embodiments 1-14, wherein the pH of the storage composition ranges from about 5.0 to about 9.0.

Embodiment 16 provides the method of any of Embodiments 1-15, wherein the at least one blood component is contacted with the adenosine and S1P at any point during a storage period of about 5 weeks to about 7 weeks.

Embodiment 17 provides the method of any of Embodiments 1-16, wherein the at least one blood component is contacted with the adenosine and S1P prior to storing the at least one blood component by cryopreservation.

Embodiment 18 provides a blood storage stock composition comprising adenosine and/or sphingosine 1-phosphate (S1P), wherein the concentration of adenosine in the stock composition is about 0.1 μM to about 10 mM and wherein the concentration of S1P in the stock composition is about 0.1 μM to about 1.2 mM.

Embodiment 19 provides the composition of Embodiment 18, further comprising at least one of NaCl, NaHCO₃, Na₂HPO₄, NaH₂PO₄, citric acid, Na-citrate, adenine, guanosine, dextrose, glucose, mannitol, gluconate, and an anti-coagulant

Embodiment 20 provides the composition of any of Embodiments 18-19, further comprising an additive agent comprising at least one of saline-adenine-glucose (SAG), saline-adenine-glucose plus mannitol (SAGM), variations of SAG/SAGM, AS-1, AS-3, AS-5, AS-7, MAP, PAGGSM, E-SOL 5, and PAG3M.

Embodiment 21 provides the composition of any of Embodiments 18-20, further comprising an anticoagulant solution comprising at least one of citrate-phosphate-dextrose (CPD), citrate-phosphate-double dextrose (CP2D), acid citrate dextrose (ACD), and citrate-phosphate-dextrose-adenine (CPDA).

Embodiment 22 provides the composition of any of Embodiments 18-21, wherein the composition is used for storing at least one blood component, wherein the at least one blood component is at least one of RBC, white blood cell, platelet, plasma derivative, and plasma.

Embodiment 23 provides the composition of any of Embodiments 18-22, wherein the pH of the composition ranges from about 5.0 to about 7.0.

Embodiment 24 provides a method of treating a subject suffering a shock due to trauma and/or hemorrhage, the method comprising administering to the subject at least one blood component and a therapeutically effective amount of the composition of any of Embodiments 18-23.

Embodiment 25 provides the method of Embodiment 24, wherein the subject is a mammal.

Embodiment 26 provides the method of any of Embodiments 24-25, wherein the subject is a human.

Embodiment 27 provides a kit comprising the stock composition of any of Embodiments 18-23 and an instructional material for use thereof, wherein the instructional material provides instructions for storing at least one blood component in a blood product.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of storing at least one blood component, wherein the method comprises contacting the at least one blood component, adenosine, and sphingosine 1-phosphate (S1P), so as to form a storage composition.

2. The method of claim 1, wherein the at least one blood component comprises at least one of a red blood cell (RBC), a white blood cell (WBC), a platelet, a plasma derivative, and plasma.

3. The method of claim 1, wherein the storage composition has at least one of:
(a) a final concentration of adenosine of about 0.01 μM to about 100 μM; and
(b) a final concentration of S1P of about 0.01 μM to about 12 μM.

4. (canceled)

5. The method of claim 1, wherein at least one of the adenosine and the S1P is contacted with the at least one blood component as a stock composition, wherein a volume of the stock composition is contacted with a volume of the storage solution, and the volume of the stock composition used for contacting the storage composition has a volume of about 1% to about 10% of the volume of the contacted storage composition.

6. The method of claim 5, wherein the stock composition has at least one of:
(a) a concentration of adenosine of about 0.1 μM to about 10 mM; and
(b) a concentration of S1P of about 0.1 μM to about 1.2 mM.

7. (canceled)

8. The method of claim 1, wherein the contacting results in at least one of:
(a) extension of an initial metabolic phase of the at least one blood component under storage; and
(b) a hypoxic-like cellular response from the at least one blood component under storage.

9. (canceled)

10. The method of claim 1, further comprising contacting the at least one blood component with an additive solution.

11. The method of claim 10, wherein the additive solution comprises at least one of an anti-coagulant agent, saline-adenine-glucose (SAG), saline-adenine-glucose plus mannitol (SAGM), variations of SAG/SAGM, AS-1, AS-3, AS-5, AS-7, MAP, PAGGSM, E-SOL 5, and PAG3M.

12. (canceled)

13. The method of claim 11, wherein the anti-coagulant agent comprises at least one of citrate-phosphate-dextrose (CPD), citrate-phosphate-double dextrose (CP2D), acid citrate or dextrose (ACD), and citrate-phosphate-dextrose-adenine (CPDA).

14. The method of claim 1, wherein the storage composition further comprises at least one of NaCl, NaHCO₃, Na₂HPO₄, NaH₂PO₄, citric acid, sodium citrate, adenine, guanosine, dextrose, glucose, mannitol, gluconate, and an anti-coagulant.

15. The method of claim 1, wherein the storage composition has a pH which has a range of about 5.0 to about 9.0.

16. The method of claim 1, wherein the at least one blood component, adenosine, and S1P are contacted at any point during a period of storage of the at least one blood component having a duration of about 5 weeks to about 7 weeks or prior to storing the at least one blood component by cryopreservation.

17. (canceled)

18. A blood storage stock composition comprising at least one of adenosine and sphingosine 1-phosphate (S1P), having at least one of:
(a) a concentration of adenosine in the stock composition of about 0.1 μM to about 10 mM; and
(b) a concentration of S1P in the stock composition of about 0.1 μM to about 1.2 mM.

19. The composition of claim 18, further comprising at least one of NaCl, NaHCO₃, Na₂HPO₄, NaH₂PO₄, citric acid, sodium citrate, adenine, guanosine, dextrose, glucose, mannitol, gluconate, and an anti-coagulant.

20. The composition of claim 18, further comprising at least one of:
(a) an additive agent comprising at least one of saline-adenine-glucose (SAG), saline-adenine-glucose plus mannitol (SAGM), variations of SAG/SAGM, AS-1, AS-3, AS-5, AS-7, MAP, PAGGSM, E-SOL 5, and PAG3M; and
(b) an anticoagulant solution comprising at least one of citrate-phosphate-dextrose (CPD), citrate-phosphate-double dextrose (CP2D), acid citrate dextrose (ACD), and citrate-phosphate-dextrose-adenine (CPDA).

21. (canceled)

22. The composition of claim 18, further comprising at least one blood component selected from the group consisting of a RBC, a WBC, a platelet, a plasma derivative, and plasma.

23. The composition of claim 18, wherein the composition has a pH which has a range of about 5.0 to about 7.0.

24. A method of treating a subject suffering a shock due to trauma or hemorrhage, the method comprising administering to the subject at least one blood component and a therapeutically effective amount of the composition of claim 18.

25. The method of claim 24, wherein the subject is a mammal, which is optionally human.

26. (canceled)

27. A kit comprising the blood storage stock composition of claim 18 and an instructional material for use thereof, wherein the instructional material provides instructions for storing at least one blood component in a blood product.
